(19) United States
(12) Patent Application Publication
(10) Pub. No.: US 2009/0281475 A1
(43) Pub. Date: Nov. 12, 2009

(54) IONTOPHORETIC ELECTROTRANSPORT DEVICE

(75) Inventors: Giovanni Nisato, Eindhoven (NL); Marc Wilhelmus Gijsbert Ponjee, Tilburg (NL); Mark Thomas Johnson, Veldhoven (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/305,381

(22) PCT Filed: Jun. 11, 2007
(86) PCT No.: PCT/IB07/52197

§ 371 Date: Dec. 18, 2008

(30) Foreign Application Priority Data
Jun. 22, 2006 (EP) 06115858.0

(51) Int. Cl. A61N 1/30

(52) U.S. Cl. 604/20

(57) ABSTRACT

An electrotransport device for transdermal drug delivery has a number of electrodes and driving circuitry for supplying driving signals to the number of electrodes. The electrodes are connected to the driving circuitry in rows and columns. The driving circuitry has row driving circuitry for supplying a row signal to a row of electrodes, and column driving circuitry for supplying a column signal to a column of electrodes. A predetermined electrode is individually addressable by supplying a row signal to a corresponding row of electrodes and a column signal to a corresponding column of electrodes.

### FIELD OF THE INVENTION

The present invention relates to transdermal drug delivery. In particular, the present invention relates to an iontophoretic electrotransport device for delivering a drug through the skin.

### BACKGROUND OF THE INVENTION

Transdermal drug delivery is an effective method of drug administration with a number of advantages over traditional oral or infusion/injection administration. For transdermal drug delivery it is necessary to overcome a barrier of the skin against the penetration of substances. Further, the barrier should be overcome in a safe and reversible way. The above-mentioned advantages of transdermal drug delivery over oral or infusion/injection administration include, among others, avoiding gastrointestinal distress; avoiding hepatic first pass effect; allowing effective use of drugs with a short therapeutic half life; enabling a controlled and sustained drug delivery; allowing rapid discontinuation in case of adverse reactions; and an increased patient compliance.

The vast majority of transdermal products currently available are passive patches and gels. However, the medical need to deliver an extended range of drugs transdermally requires a shift from passive patches to devices actively enabling controlled drug delivery. An active delivery technology potentially enables the use of smart electronics for controlled (e.g. timed) drug delivery, possibly in a closed loop system.

A known active delivery method is iontophoresis. In iontophoresis, an electric field is used to enhance the transport of (primarily charged) drug molecules across the skin barrier. In FIG. 1, a prior art iontophoretic device 1 is illustrated. The iontophoretic device 1 consists of a current source CS, an anodal electrode compartment AN and a cathode electrode compartment CA to be placed on a skin SK. The compartments AN, CA are separated from each other. A formulation with an ionized drug D+ and its counter-ion A− is placed in one of the electrode compartments, in the illustrated case in the anodal compartment AN, in particular in the compartment bearing the same charge.

A commonly used electrode pair is an Ag/AgCl pair, as is illustrated. The electrochemistry occurring at the Ag anode requires the presence of Cl− ions in the formulation in the anodal compartment. These ions may be provided by addition of NaCl molecules to the formulation. The Cl− ions present in the anodal compartment AN react with the Ag molecules to form AgCl while releasing an electron e−. In order to maintain electroneutrality in the anodal compartment AN, either a cation must move out of the anodal compartment AN and into the skin SK or an anion must leave the skin SK and enter the anodal compartment AN.

At the cathode CA, AgCl is reduced by electrons from the current source CS to form metallic Ag and a Cl− ion is released in the formulation present in the cathode compartment CA. Again, to maintain electroneutrality in the cathode compartment, either an anion has to move out of the cathode compartment CA and into the skin SK or a cation has to enter the cathode compartment CA. The electrical circuit is completed by the ions present in the skin SK, mainly Na+ and Cl−.

When a current I is applied by the current source CS, an electric field drives the positively charged molecules Na+, D+ from the anodal compartment AN through the skin SK towards the cathode compartment CA. The negatively charged molecules Cl−, A− are driven in the opposite direction.

A total electrophoretic flux is formed by two transport mechanisms: electromigration and electro-osmosis. Electromigration refers to a movement of ions in the presence of an electric field, and is proportional to an applied current density. Electro-osmosis refers to a volume flow induced by a current flow. At the molecular level, electro-osmosis can be viewed as resulting from the fact that the skin SK has an isoelectric point (pI) of about 4. As a consequence, the skin SK becomes negatively charged at a physiological acidity (pH value). Application of an electric field across such a charged membrane favors the movement of counter-ions in order to neutralize the membrane charge, which, in the case of skin, gives rise to its cation permselectivity. This in turn results in a solvent flow in the anode-to-cathode-direction. This means that (i) cations benefit from a second driving force in addition to electromigration and (ii) neutral molecules can be delivered by anodal iontophoresis.

A known iontophoretic device is powered by a constant current source to ensure that the current is kept at a desired level despite differences in skin impedance among individuals. It has been found in such an iontophoretic device that skin irritation relates to the current density of the applied current. A current density below a current density threshold of 200 μA/cm² is considered generally as being non-irritating. A current density above that current density threshold often results in skin irritation. Above a current density of 500 μA/cm² a pain is typically noticed. It has been found that, due to considerable variations in skin impedance, variations in current density as high as 10 to 1 may occur, usually causing skin irritation or burns in a more conductive area of the skin.

To overcome this problem, it is known, e.g. from U.S. Pat. No. 5,310,403 and U.S. Pat. No. 4,211,222, to use an array of electrodes in an iontophoretic device. In such devices at least one of the electrodes comprises a number of segmented electrodes. U.S. Pat. No. 4,211,222, amongst others, discloses the use of conventional electrode arrays, e.g. a plurality of positive and negative electrodes. However, these electrodes do not prevent excessive current being drawn through the skin from portions of the electrode contacting areas of the skin which have a significantly lower skin impedance than other areas.

U.S. Pat. No. 5,310,403 discloses an iontophoretic device having a pair of electrodes in which the current density of the applied current remains substantially constant over the entire area of the electrodes. The device comprises at least one segmented electrode and a current delivery circuit. However, the constant current circuit formed per each divided electrode, thus limiting the method of electrification, makes the construction of the apparatus complicated and poses cost problems.

A problem of the prior art is that one external electrical connection is required for each electrode (or set of electrodes) to control the local current densities. Consequently, the number of compartments is limited, since the number of compartments that can be realized on a single device is limited as the space required for the electrical connections becomes prohibitive.

Besides the use of segmented electrodes and corresponding current delivery circuitry, it is also known to reduce skin irritation during electrotransport delivery by delivery of an anti-inflammatory agent to reduce body irritation associated with the applied level of electric current. For this purpose, the use of a plurality of drug reservoirs (compartments) is known.

Further, besides the delivery of drugs and anti-inflammatory agents, it is also desired to release multiple types of drugs and/or chemical skin penetration enhancers. Hence, besides the need for segmented electrodes to reduce skin irritation, also an array of reservoirs/compartments that are individually controllable in parallel is desired to provide the possibility to release more than one chemical.

### OBJECT OF THE INVENTION

It is an object of the present invention to provide an electrotransport device, in particular an iontophoretic transdermal drug delivery device, having a relatively large number of individually controllable compartments.

### SUMMARY OF THE INVENTION

In an aspect, the present invention provides an electrotransport device for transdermal drug delivery, the electrotransport device comprising a number of electrodes and driving circuitry for supplying driving signals to the number of electrodes, the electrodes being connected to the driving circuitry in rows and columns, the driving circuitry comprising: row driving circuitry for supplying a row signal to a row of electrodes; and column driving circuitry for supplying a column signal to a column of electrodes, such that a predetermined electrode is individually addressable by supplying a row signal to a corresponding row of electrodes and a column signal to a corresponding column of electrodes. It is observed that the electrotransport device may further comprise a second number of electrodes (i.e. common electrodes or other electrodes which need not be connected in the form of a matrix).

In an embodiment, the present invention provides an electrotransport device for transdermal drug delivery. The electrotransport device comprises an array of drug delivery elements and driving circuitry. The array of drug delivery elements comprises at least one anodal compartment; at least one cathode compartment; at least one current source; and a number of electrodes which are distributed over the at least one anodal compartment and the at least one cathode compartment for providing at least one anode and at least one cathode and which are connectable to the power source for generating a current between the anode and the cathode. The driving circuitry is configured for supplying driving signals to the number of electrodes. The electrodes are connected to the driving circuitry in rows and columns. The driving circuitry comprises row driving circuitry for supplying a row signal to a row of electrodes; and column driving circuitry for supplying a column signal to a column of electrodes. A predetermined pair of electrodes, comprising an anode and a cathode, is addressable by supplying a row signal to a corresponding row of electrodes and a column signal to a corresponding column of electrodes.

Unlike the prior art, in which each drug delivery element of an array of drug delivery elements was provided with a separate set of wires connecting it to control circuitry, in the electrotransport device according to the present invention, the drug delivery elements are operatively arranged in rows and columns. By supplying a row signal to a single row and a column signal to a single column, only the single drug delivery element that is a part of both said single row and said single column is addressed. Thus, each drug delivery element is individually controllable.

It is noted that a row of electrodes may comprise one or more electrodes and a column of electrodes may comprise one or more electrodes. Further, functionally, the rows and columns are interchangeable. So, when a function of the electrotransport device is described or claimed in relation to a row or a column, the function may as well be provided by a column or a row, respectively.

The electrotransport device according to the present invention thus employs a matrix technology and preferably an active matrix topology as is known e.g. in the art of driving an array of liquid crystals in a display device (LCD). The electrotransport device according to the present invention may be manufactured using large-area electronics technologies, such as a-Si, LTPS or organic transistor technologies, as known in the art. Various substrates may be used, such as glass or suitable plastics. In particular, a known manufacturing process referred to as EPLAR may be used to manufacture the electrotransport device on a flexible substrate or a conformal substrate, which is advantageous for use on the skin of a patient.

The electrotransport device according to the present invention enables an electrotransport device having a large number of individually controllable electrodes, such as a number in the order of 10³-10⁶. The large number of individually controllable electrodes enables drug delivery rate control by controlling a current density per electrode as an anode or cathode of a drug delivery element. The individually controllable electrodes may be used such that substantially a same amount of current flows through each electrode independent of the impedance of the skin of the patient.

The active matrix topology allows an effective device area, i.e. the area of the device used for actual drug delivery with respect to a total device area, to be increased, which is advantageous as the rate of drug delivery may thus be improved by increasing a contact area instead of the current density, since an increase in current density may cause skin irritation.

In an embodiment, the anodal compartment and/or the cathode compartment comprises a number of reservoirs for releasably holding a drug. Each reservoir is connected to at least one electrode enabling individual control of each reservoir for releasing the drug into the respective compartment. Thus, a number of different drugs and/or other chemicals, such as an anti-inflammatory agent, a permeation enhancer, may be released from a number of individual reservoirs, i.e. release compartments. A number of techniques to control the reservoirs are available. For example, a thin lid sealing an enclosed volume of chemicals may be opened using a voltage potential or a current. Alternatively, the reservoir may comprise a gel, such as a chemically cross-linked polyelectrolyte (e.g. polyacrylic acid salt), that, similarly to a sponge, holds a chemical of interest. Upon application of a voltage or a current signal, the gel may be ‘squeezed’ to release at least a part of the chemical so that it becomes available in the anodal or cathode compartment for delivery. As electrolysis can occur near the electrodes, an AC electric field is preferable. Another mechanism is the variation of a solvent/polymer interaction parameter upon temperature variation, which in turn may be caused by an application of a voltage or current signal. Typically, upper critical solution temperature (UCST) cross-linked polymer systems are used in which the gel de-swells and expels solvent upon an increase of the temperature. Thus, an electrical signal may determine an amount of the chemical to be released.

The active matrix topology may as well be advantageously employed in other kinds of electrotransport devices comprising a relatively large number of electrodes, such as an electrotransport device using pulsed voltage or current sources to control drug delivery or a percutaneous electrode array in which electrical energy such as an electrical field or an electric current is used to promote transdermal transportation of chemicals or fluids into or out of a patient body.

### BRIEF DESCRIPTION OF THE DRAWINGS

Hereinafter, the present invention and further advantageous features are described and elucidated in more detail with reference to the appended drawings illustrating non-limiting embodiments, wherein

FIG. 1 schematically shows a prior-art iontophoretic device;

FIGS. 2A-2B schematically show a top view of a first and a second embodiment, respectively, of an electrotransport device according to the present invention;

FIGS. 2C-2D schematically show a cross sectional view of the first and the second embodiment of an electrotransport device according to FIGS. 2A-2B, respectively;

FIGS. 3A-3B schematically show a top view of a third and a fourth embodiment, respectively, of an electrotransport device according to the present invention;

FIGS. 3C-3D schematically show a cross sectional view of the third and the fourth embodiment of an electrotransport device according to FIGS. 3A-3B, respectively;

FIG. 4 schematically illustrates an active matrix topology for use in an electrotransport device according to the present invention;

FIG. 5 schematically illustrates a first embodiment of a control circuit for use in an active matrix topology according to FIG. 4;

FIG. 6 schematically illustrates a second embodiment of a control circuit for use in an active matrix topology according to FIG. 4;

FIG. 7 schematically illustrates a third embodiment of a control circuit for use in an active matrix topology according to FIG. 4;

FIG. 8 schematically illustrates a fourth embodiment of a control circuit for use in an active matrix topology according to FIG. 4;

FIG. 9A schematically shows a top view of a fifth embodiment of an electrotransport device according to the present invention; and

FIG. 9B schematically shows a cross sectional view of the fifth embodiment of an electrotransport device according to FIG. 9A.

### DETAILED DESCRIPTION OF EXAMPLES

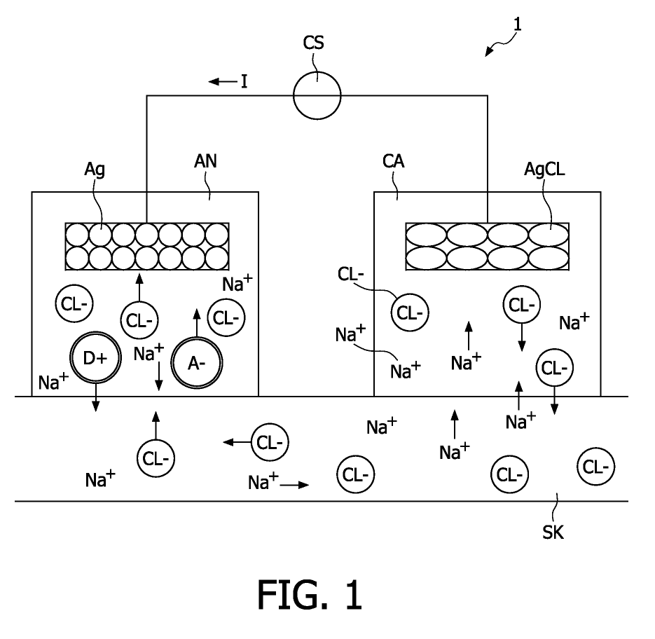

In the drawings, like reference numerals refer to like components. FIG. 1 illustrates a prior-art iontophoretic device 1 as described in detail above. Below, the present invention is elucidated with reference to the iontophoretic device 1. However, the present invention, in particular the use of an active matrix topology, is also applicable to other electrotransport devices, as mentioned above.

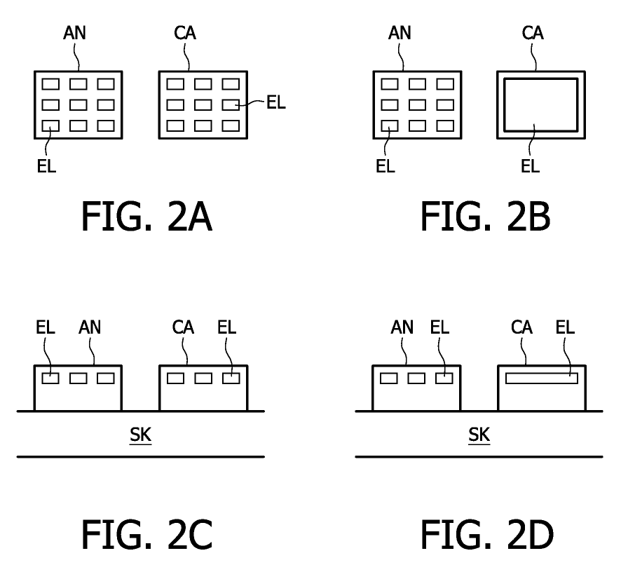

FIG. 2A shows a top view of an anodal compartment AN and a cathode compartment CA. The anodal and cathode compartments AN, CA are part of a first embodiment of an iontophoretic device as illustrated in FIG. 1. Each compartment AN, CA comprises a number of electrodes EL. FIG. 2C shows the first embodiment in a cross sectional side view and positioned on skin SK of a patient.

FIG. 2B shows a top view of an anodal compartment AN and a cathode compartment CA. The anodal and cathode compartments AN, CA are part of a second embodiment of an iontophoretic device as illustrated in FIG. 1. The anodal compartment AN comprises a number of electrodes EL. The cathode compartment comprises one electrode EL functioning as the cathode for each anodal electrode EL positioned in the anodal compartment AN. FIG. 2D shows the second embodiment in a cross sectional side view and positioned on skin SK of a patient. It is noted that, similarly, the cathode compartment CA may comprise a number of electrodes EL and the anodal compartment AN comprises a single electrode EL.

In the embodiments of FIGS. 2A-2D, the chemical to be delivered is present in at least one of the compartments AN, CA. The number of electrodes EL may be provided, for example, to enable control of a drug delivery rate and/or a current density, as mentioned above. To this end, each electrode EL is individually controllable for generating or not generating a current.

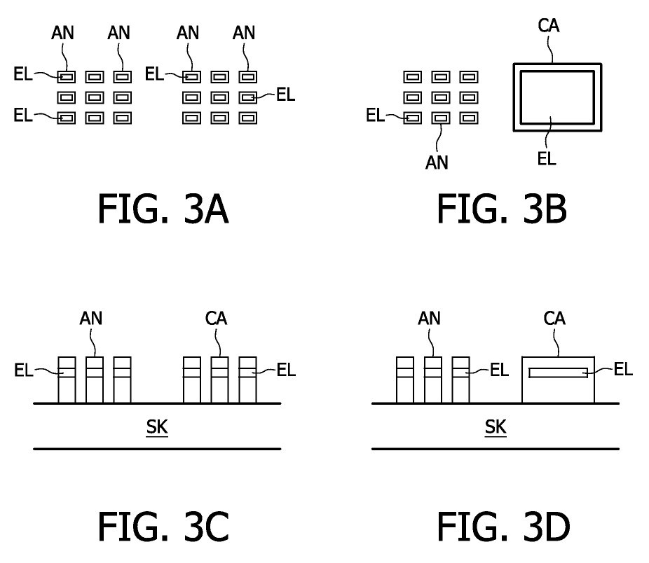

FIG. 3A shows a top view of an array of anodal compartments AN and an array of cathode compartments CA (not shown in the drawing (?)). The anodal and cathode compartments AN, CA are part of a third embodiment of an iontophoretic device as illustrated in FIG. 1. Each compartment AN, CA comprises at least one electrode EL. FIG. 3C shows the third embodiment in a cross sectional side view and positioned on skin SK of a patient.

FIG. 3B shows a top view of an array of anodal compartments AN and a cathode compartment CA. The anodal and cathode compartments AN, CA are part of a fourth embodiment of an iontophoretic device as illustrated in FIG. 1. The anodal compartments AN each comprise at least one electrode EL. The cathode compartment CA comprises one (as illustrated) or more (cf. FIG. 2A) electrodes EL functioning as the cathode for each anodal electrode EL positioned in the anodal compartments AN. FIG. 2D shows the fourth embodiment in a cross sectional side view and positioned on skin SK of a patient. It is noted that, similarly, the cathode compartment CA may comprise an array of compartments CA each comprising at least one electrode EL, and the anode may be formed in a single anodal compartment AN comprising at least one electrode EL.

In the embodiments of FIGS. 3A-3D, the chemical to be delivered is present in at least one of the compartments AN, CA. Since there are a number of anodal compartments An and/or a number of cathode compartments CA, a number of different chemicals, e.g. drugs, may be transdermally delivered by individual control of each electrode in each compartment. Thus, the number of compartments AN, CA and corresponding electrodes EL may be provided, for example, to enable control of a drug delivery rate and/or a current density, as mentioned above, and/or to enable separate control of the delivery, either sequentially or simultaneously, of different drugs. For example, a first drug may be delivered a predetermined time period after delivery of a second drug.

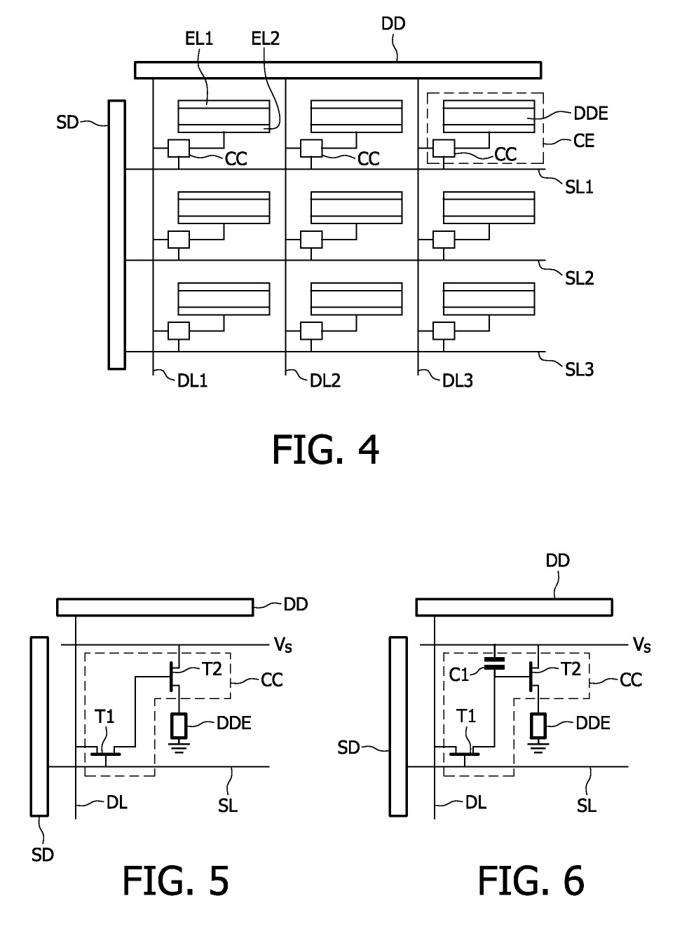

FIGS. 4-8 illustrate in more detail an active matrix topology and control for use with embodiments of the present invention, e.g. the four embodiments illustrated in FIGS. 2A-3D.

FIG. 4 shows an embodiment of an active matrix topology comprising a select driver circuit SD, a data driver circuit DD and a number of cells CE, each comprising a control circuit CC and a drug delivery element DDE comprising a first electrode EL1 and a second electrode EL2. Each cell CE, in particular each control circuit CC, is connected to one of a number of select lines SL1-SL3 and one of a number of data lines DL1-DL3. The number of select lines SL1-SL3 connect the cells CE and the select driver circuit SD to one another. The number of data lines DL1-DL3 connect the cells CE and the data driver circuit DD to one another.

As illustrated, the drug delivery elements DDE are arranged in rows and columns. A select signal generated by the select driver circuit SD and supplied on a first select line SL1 is thus supplied to each control circuit CC of a first row of cells CE. Similarly, a data signal generated by the data driver circuit DD and supplied on a first data line DL1 is thus supplied to each control circuit CC of a first column of cells CE. However, the control circuit CC is designed such that only if both a select signal and a data signal are supplied, the control circuit CC actually receives the data signal. Since only one cell CE is connected to both said first select line SL 1 and said first data line DL 1, only said one cell CE will receive the data signal on data line DL1. Thus, each drug delivery element DDE is individually addressable.

In an embodiment, each control circuit CC comprises a switch element. The switch element is operated by a select signal on a corresponding select line SL. Thus, if a select signal is supplied to the corresponding select line SL, the switch element is switched conductive, thereby providing an electrical connection between the drug delivery element DDE and the corresponding data line DL. Thus, a data signal supplied on the corresponding data line DL is supplied to the drug delivery element DDE. The data signal may, for example, be a current to be supplied to the second electrode EL2 of the drug delivery element DDE, or it may be a suitable voltage signal. If other drug delivery elements DDEs attached to the same select line SL do not need to be activated, they should receive a zero data signal. The switch element may be a transistor, diode or MIM diode device, for example.

In a further embodiment, each control circuit CC comprises two switch elements, e.g. arranged in a DRAM type of circuit. One switch element is operated by a select signal on a corresponding first select line SL. Another switch element is operated by a select signal on a corresponding second select line SL. Thus, if a select signal is supplied to the corresponding two select lines SL, the switch elements are switched conductive, thereby providing an electrical connection between the drug delivery element DDE and the corresponding data line DL. Thus, a data signal supplied on the corresponding data line DL is supplied to the single drug delivery element DDE. The data signal may, for example, be a current to be supplied to the second electrode EL2 of the drug delivery element DDE, or it may be a suitable voltage signal. The switch elements may be transistors, diodes or MIM diode devices, or any combination thereof, for example.

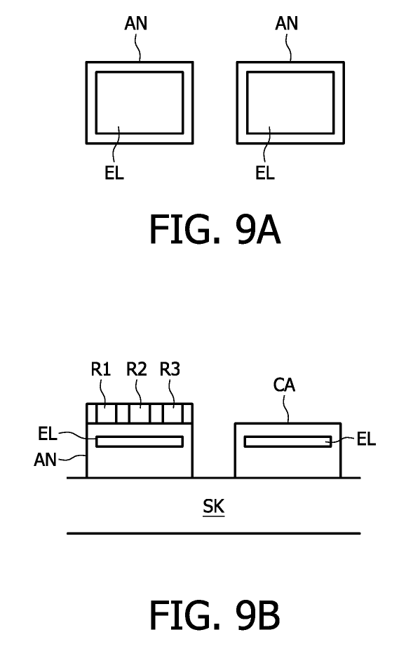

The drug delivery element DDE comprises an electrotransport system for (transdermal) drug delivery, such as an iontophoretic system as mentioned above, and may comprise additional actuating or sensing systems. The drug delivery element DDE may also comprise chemical (e.g. drug) reservoirs that can be reversibly or irreversibly released (as is explained below in relation to FIGS. 9A-9B). It is noted that in the case of iontophoresis, the skin may be considered a part of the drug delivery element DDE. It is further noted that the drug delivery element DDE may comprise a number of components, which may be both active, e.g. transistors, diodes, or passive, e.g. resistors, capacitors, electrodes. In addition, it is noted that the control circuits may comprise a number of components, which may be active and/or passive.

The select driver circuit SD and/or the data driver circuit DD may be capable of providing, if desired, signals simultaneously to one or more select lines SL or data lines DL, respectively. In an embodiment, a simpler driver circuit having a function of a de-multiplexer may be employed. The driver circuit, for example the data driver circuit DD, may then comprise a data signal generation circuit and a demultiplxer circuit. A single data signal may be supplied to the demultiplexer circuit. The demultiplexer circuit routes the signal to one of the data lines DL1-DL3, thereby only activating the drug delivery element DDE connected to the select line SL supplying a select signal and connected to said one of the data lines DL1-DL3.

Above, it is considered to provide an electrical signal for each drug delivery element DDE, i.e. a current for the electrode EL2 of an iontophoretic system, as a data signal. Thus, a data driver circuit DD can only activate a single drug delivery element DDE at a time. Consequently, drug delivery elements DDE attached to a same data driver circuit can only be activated sequentially. This makes it difficult to maintain steady delivery rates. Furthermore, if a driving current is required, it may not be possible to bring the current from the data driver circuit to the drug delivery element DDE without a loss of current due to leakage effects.

For this reason, a first embodiment of the control circuit CC, as illustrated in FIG. 5, comprises an integrated current source based on active matrix technology. The control circuit CC comprises a first select transistor T1 and a local current source embodied as a second transistor T2. A gate of the first transistor T1 is connected to the select driver circuit SD through a select line SL. A source of the first transistor T1 is connected to the data driver circuit DD through a data line DL. The drain of the first transistor T1 is connected to the gate of the second transistor T2. The source of the second transistor T2 is connected to a power supply voltage Vs. The drain of the second transistor T2 is connected to an electrode of the drug delivery element DDE.

A current flowing through the second transistor T2 from the power supply voltage Vs to the drug delivery element DDE is defined by a voltage at the gate of the second transistor T2, i.e. a transconductance of the transistor is defined by

I=α(Vₛ−V_{gate}−Vₜ)² (eq. 1)

wherein I is the transconductance, α is a constant, V_{gate} is a voltage at the gate of the second transistor T2 and Vₜ is the threshold voltage of the second transistor T2.

In operation, when a select signal is supplied at the select line SL, the first transistor T1 is conductive, thereby electrically connecting the data line DL and the gate of the second transistor T2. Thus, a current through the second transistor T2 to the drug delivery element DDE may be controlled by the voltage supplied at the data line DL as the voltage at the data line DL determines the voltage at the gate of the second transistor T2. Thus, in the present embodiment, the data signal is a voltage signal indicating an amount of current to be supplied by the second transistor T2 to the drug delivery element DDE.

In the above-described embodiments, a drug delivery element DDE is only activated when the select signal and the data signal are supplied. However, it is advantageous to incorporate a memory device into the control circuit CC, e.g. a capacitor element, or a transistor-based memory element, thereby enabling to store the data signal after an address period is completed. Thus, it is possible to have a number of simultaneously activated drug delivery elements DDE at any point across the array. It is noted that if such a memory device is available, a separate control signal may be required to de-activate the drug delivery element DDE. Further, adding the memory element allows the driving signal supplied to the drug delivery element DDE to be applied for a longer period of time, whereby the drug delivery rate can be better controlled. FIG. 6 illustrates a control circuit CC comprising such a memory element.

The second embodiment, as illustrated in FIG. 6, is substantially similar to the first embodiment, as illustrated in FIG. 5, except for a memory element embodied as a capacitor C1. A first terminal of the capacitor C1 is connected to the power supply voltage Vs and a second terminal of the capacitor C1 is connected to the drain of the first transistor T1 and the gate of the second transistor T2.

In operation, during an address period, the voltage at the gate of the second transistor T2 is stored on the capacitor C1. When the address period has ended, i.e. the data signal and/or the select signal are no longer supplied, the voltage at the gate of the second transistor T2 is held at a substantially constant level by the voltage supplied by the capacitor C1.

As mentioned above, an electrotransport device according to the present invention may advantageously be manufactured using large-area electronics. However, such large-area electronics-based constant current source array may exhibit a non-uniformity in a performance of the active elements, e.g. transistors, across the substrate. For example, in the case of LTPS technology, it is known that both a mobility factor Mf and the threshold voltage Vt of transistors vary randomly (also for transistors situated close to each other). As an example, referring to FIG. 6, if an LTPS transistor were to be used as a localized current source based upon the transconductance circuit comprising two transistors, an output of each current source would be defined by

Iₒᵤₜ=β·Mf·(Vₛ−V_{gate}−Vₜ)² (eq. 2)

wherein Iₒᵤₜ is the output current, β is a constant, Mf is the mobility factor, V_{gate} is a voltage at the gate of the current source transistor and Vₜ is the threshold voltage of the current source transistor.

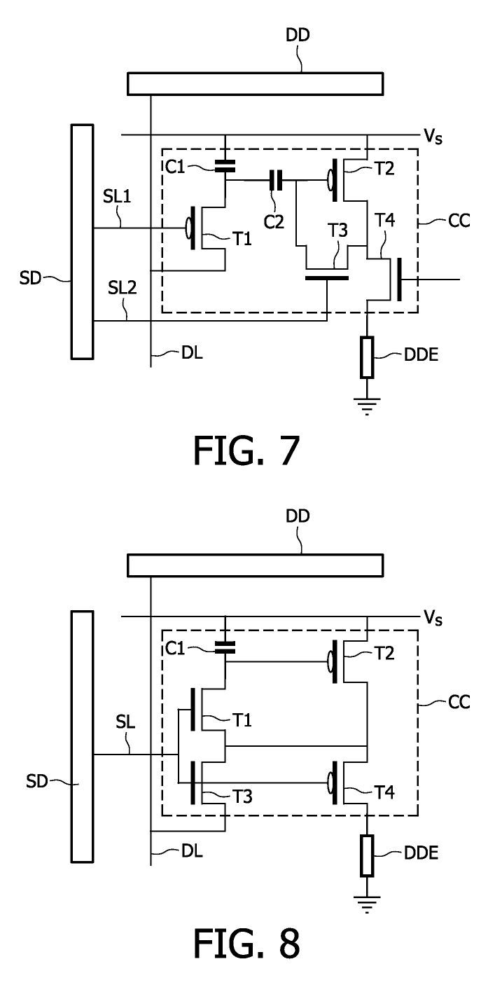

FIG. 7 illustrates a third embodiment of a control circuit CC in which the random variations of the threshold voltage Vₜ are at least partially compensated by a threshold voltage compensation circuit. It is noted that the illustrated threshold voltage compensation circuit is merely an exemplary embodiment. Other suitable circuits are known in the art and may be employed as well.

The third embodiment illustrated in FIG. 7 comprises a first transistor T1, a gate of which is connected to a first select line SL1 and a source of which is connected to a data line DL; a second transistor T2, a source of which is connected to a power supply voltage Vs; a third transistor T3, a gate of which is connected to a second select line SL2, a source of which is connected to a gate of the second transistor T2 and a drain of which is connected to a drain of the second transistor T2; and a fourth transistor T4, a gate of which is connected to a third select line SL3, a source of which is connected to the drain of the second transistor T2 and a drain of which is connected to an electrode of a drug delivery element DDE. Further, the third embodiment comprises a first capacitor C1 connected between the power supply voltage Vs and a drain of the first transistor T1 and a second capacitor C2 connected between the drain of the first transistor T1 and the gate of the second transistor T2.

In operation, a reference voltage, such as the power supply voltage Vs, is supplied on the data line DL, while the first transistor T1 and the third transistor T3 are switched conductive by suitable select signals on the first and the second select line SL1 and SL2, respectively. Then, the fourth transistor T4 is pulsed by a suitable select signal on the third select line SL3, thereby switching the second transistor T2 conductive. After the pulsed select signal on the third select line SL3, the second transistor T2 charges the second capacitor C2 up to the threshold voltage Vt of the second transistor T2. Switching the third transistor T3 non-conductive by changing the select signal on the second select line SL2 causes the threshold voltage Vt of the second transistor T2 to be stored on the second capacitor C2.

When the threshold voltage Vt of the second transistor T2 is stored on the second capacitor C2, the reference voltage of the data line DL is changed to the data signal, i.e. a data voltage. When the data voltage is applied, the data voltage is stored on the first capacitor C1. Consequently, the gate-source voltage of the second transistor T2 is substantially equal to the data voltage, as stored on the first capacitor C1, plus the threshold voltage Vt of the second transistor T2, as stored on the second capacitor C2. The current supplied by the second transistor T2 is proportional to the gate-source voltage minus the threshold voltage Vt squared (see Eq. 2). Thus, the output current is independent of the threshold voltage Vt, as the threshold voltage Vt is eliminated from the equation by first storing the threshold voltage Vt on the second capacitor C2.

FIG. 8 illustrates a fourth embodiment of a control circuit CC comprising both a threshold voltage compensition circuit and a mobility factor compensation circuit for at least partially compensating for a non-uniformity in the threshold voltage Vt and the mobility factor Mf of a current source transistor. It is noted that the illustrated threshold voltage compensition circuit and mobility factor compensation circuit are merely an exemplary embodiment. Other suitable circuits are known in the art and may be employed as well.

The fourth embodiment illustrated in FIG. 8 comprises a first transistor T1, a gate of which is connected to a select line SL; a second transistor T2, a source of which is connected to a power supply voltage Vs, a gate of which is connected to a drain of the first transistor T1, and a drain of which is connected to a source of the first transistor T1; a third transistor T3, a gate of which is connected to the select line SL, a drain of which is connected to a source of the first transistor T1 and a source of which is connected to a data line DL; and a fourth transistor T4, a gate of which is connected to the select line SL, a source of which is connected to a drain of the second transistor T2, and a drain of which is connected to an electrode of the drug delivery element DDE. Further, the control circuit CC comprises a capacitor CI connected between the power supply voltage Vs and the drain of the first transistor T1.

In operation, during an address period, the first and the third transistors T1, T3 are switched conductive by a suitable select signal on the select line SL. The select signal simultaneously switches the fourth transistor T4 non-conductive. The data line DL supplies a data signal, which is a data current in the present embodiment. The data current charges the capacitor C1 up to a voltage sufficient to pass the data current through the second transistor T2. Then, the select signal on the select line SL is removed, as a result of which the first and the third transistor T1, T3 are switched non-conductive, thereby switching the fourth transistor T4 conductive. Thus, a current may pass through the fourth transistor T4 towards the drug delivery element DDE. Thus, the mobility factor Mf and the threshold voltage Vt of the current source transistor T2 are at least partially compensated, thereby causing uniform currents to be delivered to the drug delivery elements DDE.

FIGS. 9A-9B illustrate a fifth embodiment of the electrotransport device according to the present invention. In the illustrated embodiment, the anodal compartment AN comprises at least one electrode EL as an anode; the cathode compartment CA comprises at least one electrode EL as a cathode. Referring to FIG. 9B, the anodal compartment AN further comprises a number of reservoirs R1-R3. Each reservoir R1-R3 may hold a chemical, such as a drug, skin penetration enhancer, anti-inflammatory agent, and the like, for delivery to a patient body by transdermal delivery through a skin SK. In order to deliver the chemical held in a first reservoir R1, for example, the first reservoir R1 needs to release the chemical into the anodal compartment AN. Then, the chemical may be delivered through iontophoresis using the anodal compartment AN and the cathode compartment CA. In order to release the chemical, an electrical signal is supplied to the reservoir R1. To this end, the reservoir R1 comprises an electrode which may be connected to a driving circuit through an active matrix topology in accordance with the present invention. It is noted that the number of reservoirs R1-R3 may as well be provided in the cathode compartment CA, or both compartments AN, CA, depending on the chemicals to be delivered to the patient.

A number of techniques to control the reservoirs R1-R3 are available. For example, a thin lid sealing an enclosed volume of chemicals may be opened using a voltage potential or a current, thereby possibly releasing all chemical material held in the reservoir at once. Alternatively, the reservoir may comprise a gel, such as a chemically cross-linked polyelectrolyte (e.g. polyacrylic acid salt) that, similarly to a sponge, holds a chemical of interest. Upon application of a voltage or a current signal, the gel may be ‘squeezed’ to release at least a part of the chemical so that it becomes available in the anodal or cathode compartment for delivery. As electrolysis can occur near the electrodes, an AC electric field is preferable. Another mechanism is the variation of a solvent/polymer interaction parameter upon temperature variation, which in turn may be caused by the application of a voltage or current signal. Typically, upper critical solution temperature (UCST) cross-linked polymer systems are used in which the gel de-swells and expels solvent upon a temperature increase. Thus, an electrical signal may determine an amount of the chemical to be released.

1. Electrotransport device for transdermal drug delivery, the electrotransport device comprising a number of electrodes and driving circuitry for supplying driving signals to the number of electrodes, the electrodes being connected to the driving circuitry in rows and columns, the driving circuitry comprising:
row driving circuitry for supplying a row signal to a row of electrodes; and
column driving circuitry for supplying a column signal to a column of electrodes,
wherein a predetermined electrode is individually addressable by supplying a row signal to a corresponding row of electrodes and a column signal to a corresponding column of electrodes.

2. Electrotransport device for transdermal drug delivery according to claim 1, wherein the electrotransport device comprises an array of drug delivery elements, the array of drug delivery elements comprising:
at least one anodal compartment;
at least one cathode compartment;
at least one power source;
the number of electrodes being distributed over the at least one anodal compartment and the at least one cathode compartment for providing at least one anode and at least one cathode and being connectable to the power source for generating a current between the anode and the cathode; and
at least one predetermined pair of electrodes, comprising an anode and a cathode, being addressable by supplying a row signal to a corresponding row of drug delivery elements and a column signal to a corresponding column of drug delivery elements.

3. Electrotransport device according to claim 2, wherein the electrotransport device comprises a single anode and a number of cathodes or a single cathode and a number of anodes.

4. Electrotransport device according to claim 2, wherein each drug delivery element comprises control circuitry comprising a control switch, the control switch being addressable by a row signal as an address signal for switching the control switch conductive or non-conductive for, respectively, enabling or not enabling to provide a column signal as a control signal to the control circuitry of the drug delivery element.

5. Electrotransport device according to claim 4, wherein the column signal is a power signal.

6. Electrotransport device according to claim 4, wherein the control circuitry of the drug delivery element comprises a memory element for storing a control signal and enabling the drug delivery element to be active, when the drug delivery element is not addressed.

7. Electrotransport device according to claim 4, wherein the control circuitry of the drug delivery element comprises a current source element connectable to the power source and operatively connected to the control switch such that in response to a control signal the current source element supplies a current signal to an operatively connected electrode of the drug delivery element.

8. Electrotransport device according to claim 7, wherein the electrotransport device is formed as a large-area electronics device.

9. Electrotransport device according to claim 8, wherein the current source element is formed as a transistor, and the control circuitry comprises a threshold voltage compensation circuit for compensating a random variation of the threshold voltage among the transistors of the control circuitry of each drug delivery element.

10. Electrotransport device according to claim 8, wherein the current source element is formed as a transistor, and the control circuitry comprises a mobility factor compensation circuit for compensating a random variation of the mobility factor among the transistors of the control circuitry of each drug delivery element.

11. Electrotransport device according to claim 2, wherein the at least one anodal compartment and/or the at least one cathode compartment comprises a number of reservoirs for releasably holding a drug, each reservoir being connected to at least one electrode enabling individual control of each reservoir for releasing the drug into said anodal compartment or cathode compartment.
